(19) United States
(12) Patent Application Publication
(10) Pub. No.: US 2022/0204571 A1
(43) Pub. Date: Jun. 30, 2022

(54) SYSTEM AND METHOD FOR USING PEPTIDES FOR FLAVORINGS

(71) Applicant: Sophie's BioNutrients Pte. Ltd., Singapore (SG)

(72) Inventor: Yao-Hsin Wang, Singapore (SG)

(73) Assignee: Sophie's BioNutrients Pte. Ltd., Singapore (SG)

(21) Appl. No.: 17/563,094

(22) Filed: Dec. 28, 2021

Related U.S. Application Data
(60) Provisional application No. 63/132,720, filed on Dec. 31, 2020.

(51) Int. Cl. C07K 14/405; A23L 27/21; A23L 33/195; A23J 3/20; A23J 3/22; A23J 1/00

(52) U.S. Cl. CPC C07K 14/405; A23L 27/21; A23L 33/195; A23V 2002/00; A23J 3/227; A23J 1/009; A23J 3/20

(57) ABSTRACT

A method for using peptides for flavorings is described. The method includes, at least, obtaining a microalgae, extracting chlorella protein from the microalgae, modifying a factor associated with the chlorella protein to change an amino acid combination of the chlorella protein, and identifying a peptide flavoring associated with the modified amino acid combination.

### CROSS-REFERENCE TO RELATED APPLICATION SECTION

This application is a U.S. Non-Provisional patent application that claims priority to U.S. Provisional Patent Application Ser. No. 63/132,720 filed on Dec. 31, 2020, the entire contents of which are hereby incorporated by reference in their entirety.

### FIELD OF THE EMBODIMENTS

The field of the invention and its embodiments relate to a system and method for using peptides for flavoring.

### BACKGROUND OF THE EMBODIMENTS

Algae are photosynthetic organisms that grow in a range of aquatic habitats, including lakes, pounds, rivers, and oceans. Algae can tolerate a wide range of temperatures, salinities, and pH values, as well as differing light intensities. Additionally, algae may also grow alone or in symbiosis with other organisms. Algae may be broadly classified as Rhodophyta (red algae), Phaeophyta (brown algae), or Chlorophyta (green algae). Algae may be further classified by size, as macroalgae (which are multicellular, large-size algae that are visible with the naked eye) or microalgae (which are microscopic, single cells that may be prokaryotic or eukaryotic).

Currently, there are an estimated 300,000 to 1 million species of microalgae in existence. Microalgae has recently attracted considerable interest due to their extensive applications in the renewable energy field, the biopharmaceutical field, and the nutraceutical field. Specifically, microalgae may be a sustainable and economical source of biofuels, bioactive medicinal products, and food ingredients. Moreover, microalgae also have applications in wastewater treatment and atmospheric CO₂ mitigation. Thus, microalgae produces a wide range of bioproducts, including polysaccharides, lipids, pigments, proteins, vitamins, bioactive compounds, and antioxidants.

Chlorella is a genus of single-celled green algae belonging to the division Chlorophyta. Chlorella I spherical in shape, about 2 to 10 μm in diameter, and is without flagella. It contains the green photosynthetic pigments chlorophyll-a and -b in its chloroplast. Chlorella multiples rapidly, requiring only carbon dioxide, water, sunlight, and a small amount of minerals to reproduce.

Chlorella is a potential food source since it is high in protein and other essential nutrients. For example, when dried, chlorella contains about 45% protein, 20% fat, 20% carbohydrate, 5% fiber, and 10% minerals and vitamins (e.g., vitamin B12, vitamin C, iron, magnesium, zinc, copper, potassium, and/or calcium, etc.). Due to this, chlorella has been labeled as a “superfood” and has garnished significant attention from the vegan community. Further, chlorella has been explored as a potential source of food and energy because its photosynthetic efficiency can, in theory, reach 8%, which exceeds that of other highly efficient crops, such as sugar cane.

Although microalgae are feasible sources for bioenergy and biopharmaceuticals in general, some limitations remain. One such limitation to microalgae cultivation is light intensity. Light duration and intensity directly affect the photosynthesis of microalgae. At very low and very high light intensities, microalgae cannot grow efficiently. Higher light intensities will increase a photosynthetic rate of the microalgae to a maximum point, after which it levels off until the photosynthetic rate is balanced by photorespiration and photoinhibition. Photorespiration refers to a process in plant metabolism, where the enzyme, ribulose-1,5-bisphosphate carboxylase/oxygenase (RuBisCO), oxygenates ribulose 1,5-bisphosphate (RuBP), wasting some of the energy produced by photosynthesis. Photoinhibition is light-induced reduction in the photosynthetic capacity of a plant, alga, or cyanobacterium. Essentially, optimal light intensity needs to be determined experimentally in each case to maximize CO₂ assimilation and minimize both photorespiration and photoinhibition. Uniform distribution of light is also needed to avoid photoinhibition.

Another limitation to the growth of microalgae is temperature. Each species of microalgae has its own optimal growth temperature. Increasing a temperature to the optimum range exponentially increases algal growth, but an increase or decrease in the temperature beyond the optimal point retards or even stops algae growth and activity. The optimum temperature range for most algal species is 20-30° C. Growing microalgae cultures at non-optimal temperatures will result in high biomass losses.

A further limitation to microalgae growth involves ensuring the nutritional needs of the microalgae are met. Typically, all strains of microalgae have the following backbone: nitrogen, phosphorus, and carbon (CH_{1.7}O_{0.4}N_{0.15}P_{0.0094}). Some marine microalgae species also require silicon as a macronutrient. Specifically, quantities of the available nitrogen in the culture directly alter cell growth. Nitrogen limitation in the microalgae culture can reduce growth and biomass productivity, however, can increase production of carbohydrates and lipids. As an illustrative example, an optimum concentration of nitrogen for Chlorella vulgaris is 0.5 g/l, at which it produces 3.43 g/l biomass. Moreover, the micronutrients molybdenum (Mo), potassium (K), cobalt (Co), iron (Fe), magnesium (Mg), manganese (Mn), boron (B), and zinc (Zn) are only required in trace amounts, but have been shown to have a strong impact on microalgae growth, as they influence many enzymatic activities in algal cells. Nutrient deficiency greatly affects the microalgae growth rate and results in low biomass.

With increasing attention being paid to the consumption of healthy nutritional foods, algal protein has moved to the forefront of non-animal protein sources. However, the applications of chlorella protein as a functional ingredient in food still requires further exploration. Thus, a need exists for methods to modify one or more factors associated with chlorella protein to change an amino acid combination of the chlorella protein. Moreover, a need exists for methods to identify a peptide flavoring associated with the modified amino acid combination.

### SUMMARY OF THE EMBODIMENTS

The present invention and its embodiments provide a system and method for using peptides for flavoring.

An embodiment of the instant invention describes a method for using peptides for flavoring. The method includes numerous process steps, such as: obtaining a microalgae. In examples, the microalgae may be a mixotrophic strain. The strain of the microalgae may be a Botryococcus sudeticus strain, a Botryococcus strain, a Neochloris oleabundans strain, a Neochloris strain, a Chlamydomonas reinhardtii strain, or a Chlamydomonas strain, among others not explicitly listed herein. Next, the method may include extracting chlorella protein from the microalgae and modifying a factor associated with the chlorella protein to change an amino acid combination of the chlorella protein. The factor may include: a pH level of the microalgae, a wavelength of irradiance of light onto the microalgae during a fermentation process, a type of light used during the fermentation process, a feedstock for the microalgae, a carbon source of a culture media in which the microalgae is located, a growth temperature for the microalgae, a flow rate of air into a bioreactor during a fermentation process, a flow rate of air/O₂ mixtures into the bioreactor during the fermentation process, a length of a fermentation process of the microalgae, a flow rate of noble gases into the bioreactor during the fermentation process, and/or an incubation time period for the microalgae under a mixotrophic growth condition, among other factors not explicitly listed herein.

Next, the method may optionally include: adding a stimulant to the chlorella protein to modify the amino acid combination of the chlorella protein, where the stimulant is a substrate. The substrate may be: a spent grain, okara, and molasses. Then, the method may include: identifying a peptide flavoring associated with the modified chlorella protein.

### BRIEF DESCRIPTION OF THE DRAWINGS

FIG. 1 depicts a schematic block diagram of a method for using peptides for flavoring, according to at least some embodiments disclosed herein.

FIG. 2 depicts a graph of various amino acids and amounts in an example crab flavor, according to at least some embodiments disclosed herein.

FIG. 3 depicts a graph of various amino acids included in the example crab flavor, according to at least some embodiments disclosed herein.

FIG. 4 depicts a graph of various amino acids included in three examples, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

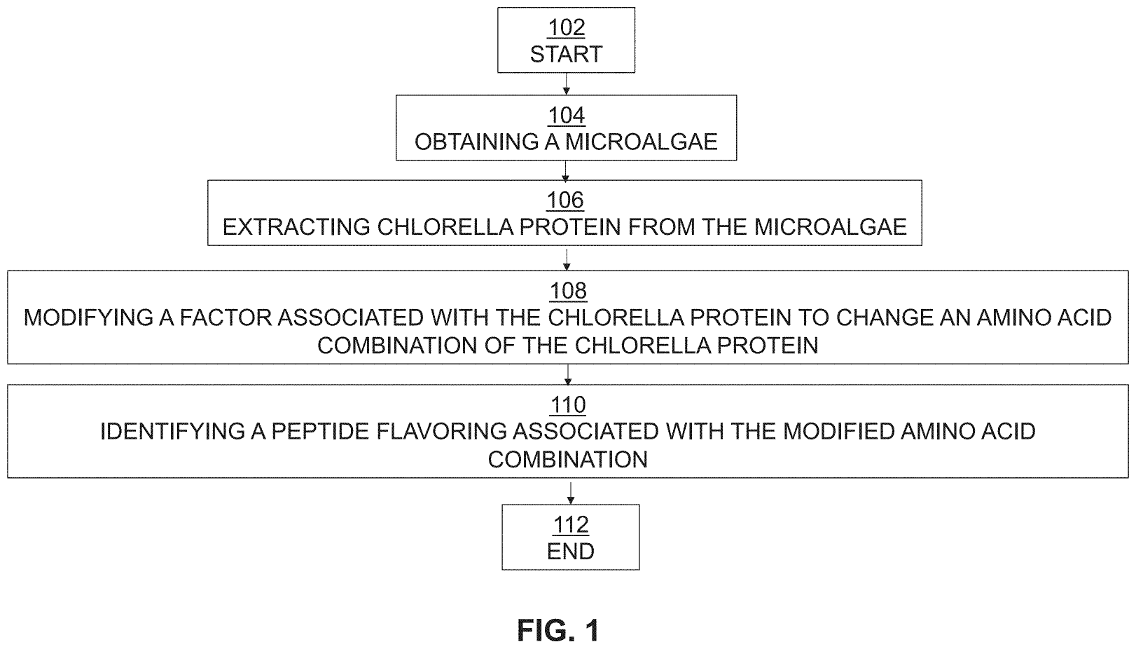

FIG. 1 depicts a schematic block diagram of a method for using peptides for flavoring, according to at least some embodiments described herein. The method of FIG. 1 may begin at a process step 102. The process step 102 may be followed by a process step 104 that includes obtaining the microalgae. As defined herein, a “microalgae” refers to a eukaryotic microbial organism that contains a chloroplast, and optionally, that is capable of performing photosynthesis, or a prokaryotic microbial organism capable of performing photosynthesis.

In some examples, the microalgae may first be cultivated in a bioreactor system or a photobioreactor system (such as a fermentation tank). As described herein, a “bioreactor” is an enclosure or partial enclosure, in which cells are cultured, and optionally in suspension. As described herein, a “photobioreactor” refers to a container, at least part of which is at least partially transparent or partially open, thereby allowing light to pass through, in which one or more microalgae cells are cultured.

A culture media may include a carbon source and may be located inside of the bioreactor system. The microalgae may be located in the culture media. A strain of the microalgae may include: a Botryococcus sudeticus strain, a Botryococcus strain, a Neochloris oleabundans strain, a Neochloris strain, a Chlamydomonas reinhardtii strain, or a Chlamydomonas strain, among others. In additional examples, the microalgae is of a mixotrophic strain. In examples, the microalgae may be adapted for both autotrophic growth and heterotrophic growth during a time period.

As defined herein, an organism capable of “autotrophic growth” is one that produces complex organic compounds using carbon from simple substances such as carbon dioxide. As defined herein, an organism capable of “heterotrophic growth” is one that cannot produce its own food. As defined herein, an organism capable of “mixotrophic growth” is one that derives nourishment from both autotrophic and heterotrophic mechanisms.

The process step 104 may be followed by a process step 106, which includes extracting chlorella protein from the microalgae. Numerous extraction methods may be used, such as mechanical grinding, high-pressure homogenization, ultrasonic treatment, pulse dyslenoid to release the protein molecules to facilitate further extraction processes like water, alkali or enzyme, and then use of isoelectric precipitation, and salting out (salt induced precipitation) methods. Additional extraction methods include: an alkaline solution extraction method, an enzyme extraction method, and a low-temperature DES extraction method, among others. It should be appreciated that further extraction methods may be used, which are not explicitly listed herein.

The process step 106 is followed by a process step 108. The process step 108 includes: modifying a factor associated with the chlorella protein to change an amino acid combination of the chlorella protein. As defined herein, an “amino acid” refers to an organic compound that contains amine (—NH2) and carboxyl (—COOH) functional groups, along with a side chain (R group) specific to each amino acid. The key elements of an amino acid are carbon (C), hydrogen (H), oxygen (O), and nitrogen (N), although other elements are found in the side chains of certain amino acids. About 500 naturally occurring amino acids are known (though only 20 appear in the genetic code) and can be classified in many ways. They can be classified according to the core structural functional groups' locations as alpha- (α-), beta- (β-), gamma- (γ-) or delta-(δ-) amino acids; other categories relate to polarity, pH level, and side chain group type (aliphatic, acyclic, aromatic, containing hydroxyl or sulfur, etc.).

In examples, the factor may include a pH level of the microalgae, a wavelength of irradiance of light onto the microalgae during a fermentation process, a type of light used during the fermentation process, a feedstock for the microalgae, a carbon source of a culture media in which the microalgae is located, a growth temperature for the microalgae, a flow rate of air into a bioreactor during a fermentation process, a flow rate of air/O₂ mixtures into the bioreactor during the fermentation process, a length of a fermentation process of the microalgae, a flow rate of noble gases into the bioreactor during the fermentation process, and/or an incubation time period for the microalgae under a mixotrophic growth condition, among others. In examples, the carbon source for the culture media may be glucose, fructose, sucrose, galactose, xylose, mannose, rhamnose, N-acetylglucosamine, glycerol, floridoside, glucuronic acid, corn starch, depolymerized cellulosic material, sugar cane, sugar beet, lactose, milk whey, or molasses, among other examples not explicitly listed herein.

As defined herein, a “feedstock” refers to what kind of food waste one uses to feed microalgae. Different feed stocks include differing nitrogen and carbon sources.

As defined herein, “fermentation” refers a metabolic process that produces chemical changes in organic substrates through the action of enzymes. In the context of food production, “fermentation” may refer to any process in which the activity of microorganisms brings about a desirable change to a foodstuff or beverage. In microorganisms, fermentation is the primary means of producing adenosine triphosphate (ATP) by the degradation of organic nutrients anaerobically. As an example, fermentation may be used to produce alcoholic beverages, such as wine and beer.

An optional process step may follow the process step 108 and may include adding a stimulant to the chlorella protein to change the amino acid combination of the chlorella protein. The stimulant is a substrate and may include a spent grain, okara, or molasses, among other examples.

It should be appreciated that changing the amino acid combination of the chlorella protein by the process step 108 and/or the optional process step described herein may result in the creation of functional proteins. Proteins are macromolecules consisting of one or more long chains of amino acid residues. Proteins perform a vast array of functions within organisms, including catalyzing metabolic reactions, DNA replication, responding to stimuli, providing structure to cells, and organisms, and transporting molecules from one location to another. Proteins differ from one another primarily in their sequence of amino acids, which is dictated by the nucleotide sequence of their genes, and which usually results in protein folding into a specific three-dimensional structure that determines its activity.

Amino acids are the basic building blocks of the body and are organic compounds that contain amine (—NH2) and carboxyl (—COOH) functional groups, along with a side chain (R group) specific to each amino acid. In the form of proteins, amino acid residues form the second-largest component (water is the largest) of human muscles and other tissues. Amino acids are extremely versatile and more than 200 different amino acids exist. The most commonly known are the 22 proteinogenic amino acids.

Amino acids prove to be beneficial in numerous fields. For example, L-methionine and L-arginine work together with Glucosamine, Chondroitin, omega-3 and Methylsulfonylmethane (MSM) to prevent and treat arthritis. L-glutamine, L-arginine and L-cysteine may be useful to improve one's immune system. Branched-chain amino acids (BCAAs) and especially L-leucine are essential for growth, recovery and maintenance of all muscle tissue. L-arginine, L-methionine, L-cysteine, L-lysine, L-glycine and L-proline boost ones natural skin and nail beauty.

L-arginine, L-carnitine and L-cysteine can significantly improve sperm quality and therefore male fertility. L-cysteine, L-glutathione and L-carnitine are powerful antioxidants, which protect ones cells from oxidative stress caused by free radicals. L-arginine and Pine bark Extract improve circulation throughout and protect ones body's arterial walls. Managing L-tryptophan levels can be good for ones sleep. BCAAs, L-glutamine and L-glycine reduce the risk of inflammatory diseases and chronic pain by strengthening ones immune system.

Magnesium, phytoestrogens and L-arginine help manage menopause by reducing hot flushes. L-arginine, L-lysine, zinc and vitamin C improve digestion and protect one from rectal diseases. L-arginine and Ginkgo biloba improve blood circulation, increasing oxygen and nutrient availability within the ear. Moreover, one may face a reduced risk of diabetes with L-arginine and L-carnitine, zinc, magnesium, chromium and omega-3. Thus, modifying the amino acid combination in the chlorella protein may result in the creation of functional proteins.

Recently, technologies have focused on gaining access to unique and high valued engineered peptide modalities. This is achieved by using novel synthetic chemical methods with peptide display technologies. As defined herein, a “peptide” is a short chain of between two and fifty amino acids, linked by peptide bonds. Chains of fewer than ten or fifteen amino acids are called oligopeptides, and include dipeptides, tripeptides, and tetrapeptides. A polypeptide is a longer, continuous, unbranched peptide chain of up to approximately fifty amino acids. Hence, peptides fall under the broad chemical classes of biological polymers and oligomers, alongside nucleic acids, oligosaccharides, polysaccharides, and others. A polypeptide that contains more than approximately fifty amino acids is known as a protein.

Peptides have a taste, covering essentially the entire range of established taste modalities: sweet, bitter, umami, sour and salty. The tastes of sour and salty are due to the presence of charged terminals and/or charged side chains, thus reflecting only the zwitterionic nature of these compounds and/or the nature of some side chains but not the electronic and/or conformational features of a specific peptide. The sweet, umami, and bitter tastes are represented by different families of peptides and have a direct relationship with food acceptance or rejection. See, Piero A. Temussi, “The Good Taste of Peptides,” J. Pept. Sci., 2012, Vol. 18, Issue 2, Pages 73-82, the entire contents of which are hereby incorporated by reference in their entirety. As such, by modification of the amino acid combinations, peptides may be derived having different flavoring components. For example, if the fermentation process of the microalgae is prolonged, the flavor profile may become fishy.

Based on this, a process step 110 may follow the process step 108 and may include identifying a peptide flavoring associated with the modified amino acid combination. Such identification may occur by any means or method known to a person having ordinary skill in the art. In some examples, the identification of the peptide flavoring may be transmitted to a device for analysis. Such device may be any device known to one having ordinary skill in the art.

Moreover, the device may compile and house a natural products library. In some examples, an analytical platform may be used that combines mass spectrometry (GC/LC-based) with organoleptics to allow for the investigation of ingredients that make up an aroma. As defined herein, “mass spectrometry” is an analytical techniques that is used to measures the mass-to-charge ratio of ions. As defined herein, “organoleptic properties” are the aspects of food, water or other substances that create an individual experience via the senses—including taste, sight, smell, and touch. In additional examples, a taste receptor platform may be used that includes cell-based assays used to identify and evaluate substances with active taste properties. These high-throughput functional cell-based assays are modular in design, allowing the rapid expression of multiple receptors of choice for use in studying tastants in an intracellular calcium release assay.

In further examples, the method of FIG. 1 may include one or more steps to predict aroma molecule absorption by different packaging polymers. Such steps may include use of a mathematical calculation to rapidly predict the extent to which packaging can absorb organic molecules from the beverage products they contain. In some examples, this process may include a combination of Flory-Higgins theory with group contribution methods (GCM). As described herein, “Flory-Huggins theory” is a lattice model of the thermodynamics of polymer solutions which takes account of the great dissimilarity in molecular sizes in adapting the usual expression for the entropy of mixing. The result is an equation for the Gibbs free energy change ΔGₘ for mixing a polymer with a solvent. As described herein, “GCM” assumes the properties of a substance are the sum of contributions from all constituent chemical groups, so the properties of the molecule CH₃—CH₂—CH₂—COOH, for example, can be calculated by summing the properties of one CH₃ group, two CH₂ groups and one COOH group. In the case of food packaging, the calculation predicts the extent to which a given aroma molecule will dissolve into a given polymer by summing the mixing of each chemical component of the polymer with each chemical component of the aroma molecule. See, “Formula for flavor,” 2018, A*STAR RESEARCH, accessible at https://research.a-star.edu.sg/articles/highlights/formula-for-flavor/ (accessed on Dec. 10, 2020), the entire contents of which are hereby incorporated by reference in their entirety.

It should be appreciated that the modified chlorella protein may be used as a protein flour with different application functions, different nutritional functions, and/or different functional properties based on the modified factor(s) and/or the applied stimulant(s). In some examples, the protein flour may be used to make a plant-based food product, such as meatloaf or tofu. Such functional properties performed by proteins in food include: solubility, water absorption and binding, viscosity, gelation, cohesion-adhesion, elasticity, emulsification, fat adsorption, flavor binding, and/or foaming, among others. For example, water absorption and binding may be significant in meats, sausages, breads, and cakes, and may be the result of hydrogen-bonding of water and entrapment of water. Additionally, viscosity may be significant for soups and gravies and may result from thickening. Gelation may be important in meats, curds, and cheeses, and may be a result of protein matrix formation and setting. As such, one can use the modified chlorella protein powder that has similar nutritional, functional, and applicational profiles to the animal protein it is trying to replace. Moreover, a process step 112 follows the process step 110 and ends the method of FIG. 1.

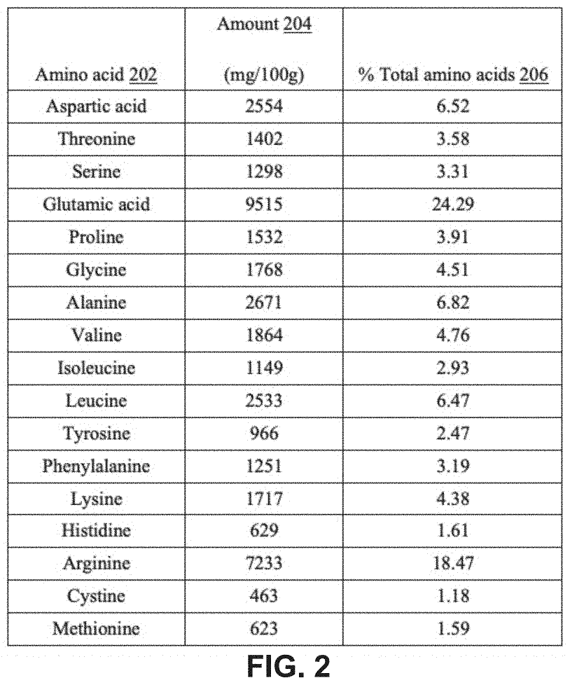

FIG. 2 depicts a graph of various amino acids and amounts in an example crab flavor, according to at least some embodiments disclosed herein. Specifically, FIG. 2 includes a first column 202 associated with a given amino acid, a second column 204 associated with an amount 204 of the given amino acid of the first column 202 in mg/100 g and a third column 206 associated with a % of the total amino acids.

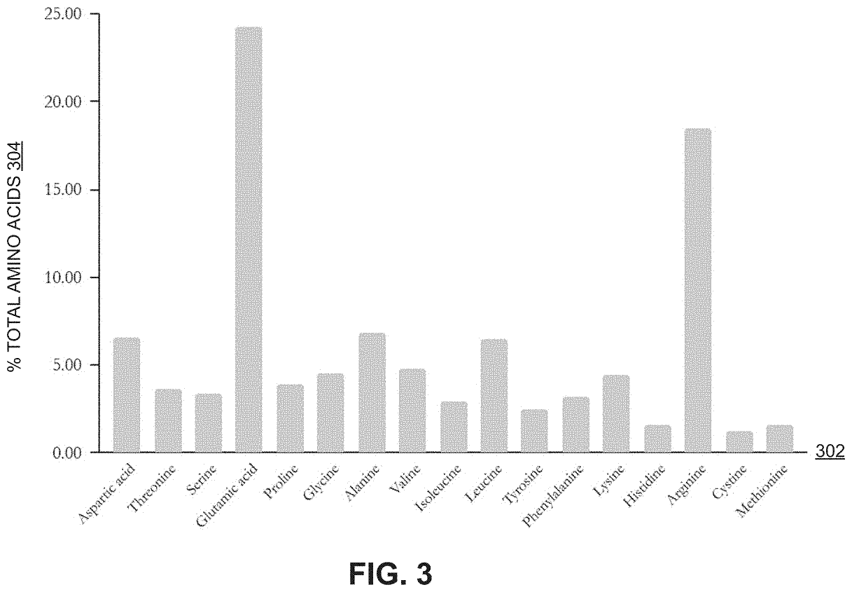

FIG. 3 depicts a graph of various amino acids included in the example crab flavor, according to at least some embodiments disclosed herein. Specifically, FIG. 3 includes an x-axis 302 associated with various amino acids and a y-axis 304 associated with % of the total amino acids.

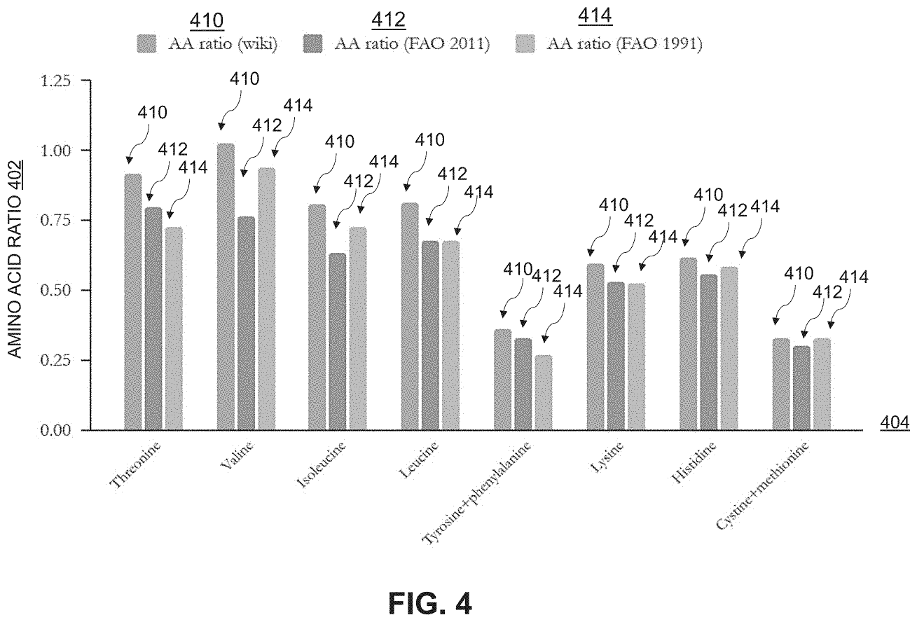

FIG. 4 depicts a graph of various amino acids included in a first example 310, a second example 312, and a third example 314, according to at least some embodiments disclosed herein. Specifically, FIG. 4 includes an x-axis 306 associated with various amino acids and a y-axis 308 associated with various amino acid ratios.

As shown in FIG. 2-FIG. 4, the example crab flavor described herein is high in glutamic acid and arginine. Specifically, arginine provides the crab/seafood flavor, while glutamic acid provides the umami flavor.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method for using peptides for flavorings, comprising:
obtaining a microalgae;
extracting chlorella protein from the microalgae;
modifying a factor associated with the chlorella protein to change an amino acid combination of the chlorella protein; and
identifying a peptide flavoring associated with the modified amino acid combination.

2. The method of claim 1, wherein a strain of the microalgae is selected from the group consisting of: a Botryococcus sudeticus strain, a Botryococcus strain, a Neochloris oleabundans strain, a Neochloris strain, a Chlamydomonas reinhardtii strain, and a Chlamydomonas strain.

3. The method of claim 1, wherein the microalgae is a mixotrophic strain of the microalgae.

4. The method of claim 1, wherein the factor is selected from the group consisting of: a pH level of the microalgae, a wavelength of irradiance of light onto the microalgae during a fermentation process, a type of light used during the fermentation process, a feedstock for the microalgae, a carbon source of a culture media in which the microalgae is located, a growth temperature for the microalgae, a flow rate of air into a bioreactor during a fermentation process, a flow rate of air/O₂ mixtures into the bioreactor during the fermentation process, a length of a fermentation process of the microalgae, a flow rate of noble gases into the bioreactor during the fermentation process, and/or an incubation time period for the microalgae under a mixotrophic growth condition.

5. The method of claim 1, further comprising:
adding a stimulant to the chlorella protein to modify the amino acid combination of the chlorella protein.

6. The method of claim 5, wherein the stimulant is a substrate, and wherein the substrate is selected from the group consisting of: a spent grain, okara, and molasses.

7. The method of claim 1, further comprising:
utilizing the modified chlorella protein as a protein flour to create a plant-based food product.

8. The method of claim 7, wherein the plant-based food product is a meatloaf or a tofu.
